(19) United States
(12) Patent Application Publication
(10) Pub. No.: US 2020/0138269 A1
(43) Pub. Date: May 7, 2020

(54) INSERTION SUPPORT DEVICE, INSERTION SUPPORT METHOD, AND ENDOSCOPE APPARATUS INCLUDING INSERTION SUPPORT DEVICE

(71) Applicant: OLYMPUS CORPORATION, Tokyo (JP)

(72) Inventor: Hirokazu NISHIMURA, Hachioji-shi (JP)

(73) Assignee: OLYMPUS CORPORATION, Tokyo (JP)

(21) Appl. No.: 16/720,110

(22) Filed: Dec. 19, 2019

Related U.S. Application Data
(63) Continuation of application No. PCT/JP2017/022830, filed on Jun. 21, 2017.

(51) Int. Cl. A61B 1/00; A61B 1/31; A61B 1/005

(52) U.S. Cl. CPC A61B 1/00055; A61B 1/00009; A61B 1/00119; A61B 1/005; A61B 1/31

(57) ABSTRACT

An insertion support device includes an image input section to which endoscopic images generated in time series are input, a situation determination section configured to determine a situation of endoscope insertion based on the inputted endoscopic images, and an information output section configured to output information corresponding to at least one operation from information corresponding to operations prepared in association with an endoscope insertion procedure, based on a determination result by the situation determination section.

### CROSS-REFERENCE TO RELATED APPLICATIONS

This application is a Continuation Application of PCT Application No. PCT/JP2017/022830, filed Jun. 21, 2017, the entire contents of which are incorporated herein by reference.

### BACKGROUND OF THE INVENTION

### 1. Field of the Invention

The present invention relates to an insertion support device configured to support insertion of an endoscope, an insertion support method, and an endoscope apparatus including the insertion support device.

### 2. Description of the Related Art

Upon colonoscopy insertion, systems and methods for performing insertion support have been suggested. For example, Japanese Patent No. 4885388 and Japanese Patent No. 5687583 disclose an endoscope insertion direction detection method and an endoscope insertion direction detection device configured to detect a lumen direction as an endoscope insertion direction by analyzing an endoscopic image. In particular, Japanese Patent No. 5687583 discloses an endoscope insertion direction detection device including a display unit configured to display a detected endoscope insertion direction.

For example, Japanese Patent No. 4855902 discloses a system including a display control unit configured to determine whether or not to cause the display unit to display insertion auxiliary information for supporting an insertion operation, that is, information corresponding to the operation, based on a display period control value calculated from the latest analysis data and past analysis data of the insertion state.

For example, Japanese Patent No. 4855912 discloses an endoscope insertion shape analysis system configured to detect an excessive extension of the large intestine and perform a smooth insertion operation of the insertion section.

### BRIEF SUMMARY OF THE INVENTION

An insertion support device according to the present invention includes an image input section to which a plurality of endoscopic images generated in time series are input, a situation determination section configured to determine a situation of endoscope insertion based on the plurality of inputted endoscopic images, and an information output section configured to output information corresponding to at least one operation from information corresponding to a plurality of operations prepared in association with an endoscope insertion procedure, based on a determination result by the situation determination section.

An insertion support method according to the present invention includes inputting a plurality of time-sequentially generated endoscopic images, determining a situation of endoscope insertion based on the plurality of inputted endoscopic images, and outputting information corresponding to at least one operation from information corresponding to a plurality of operations prepared in association with an endoscope insertion procedure, based on the determining the situation.

Advantages of the invention will be set forth in the description that follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing an example of an endoscope apparatus according to an embodiment of the present invention.

FIG. 2 is a diagram showing an example of an endoscope and an insertion shape observation device according to an embodiment of the present invention.

FIG. 3 is a diagram showing an example of a loop formation pattern of an insertion section.

FIG. 4 is a diagram showing an example of an insertion support flow.

FIG. 5 is a diagram showing an example of an insertion situation, insertion difficulty determination, and auxiliary information.

FIG. 6 is a diagram showing an example of insertion performed by a non-loop method.

### DETAILED DESCRIPTION OF THE INVENTION

Embodiments of the present invention will be explained below with reference to the drawings. Hereinafter, an example of an endoscope apparatus including a colonoscope will be explained.

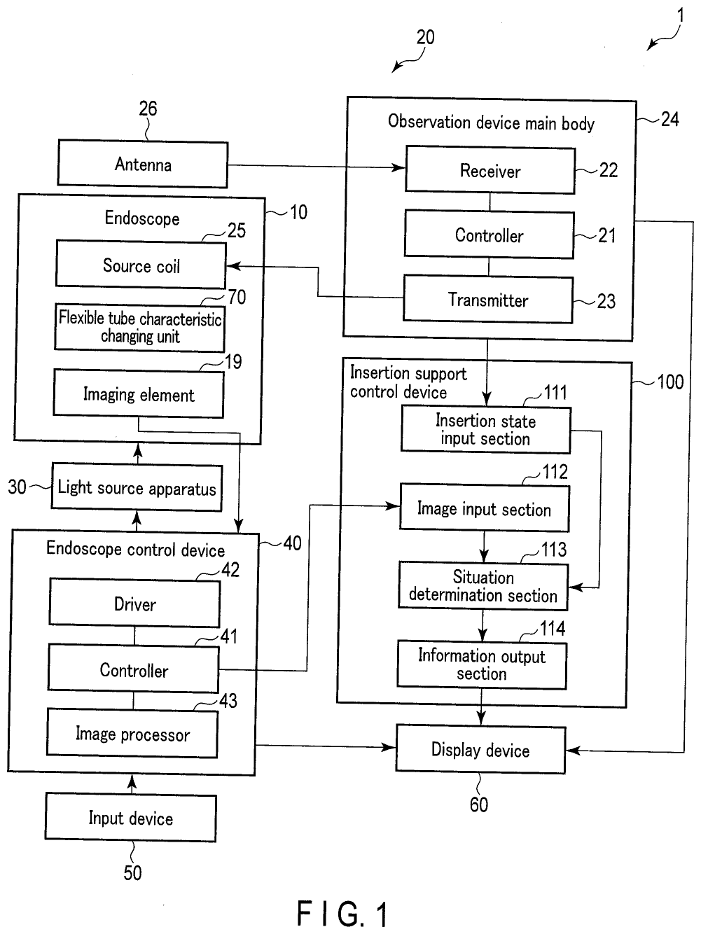

FIG. 1 is a block diagram showing an example of an endoscope apparatus 1 according to an embodiment of the present invention. Here, the endoscope apparatus 1 includes an endoscope 10, which is a colonoscope, an insertion shape observation device 20, a light source apparatus 30, an endoscope control device 40, an input device 50, a display device 60, and an insertion support control device 100.

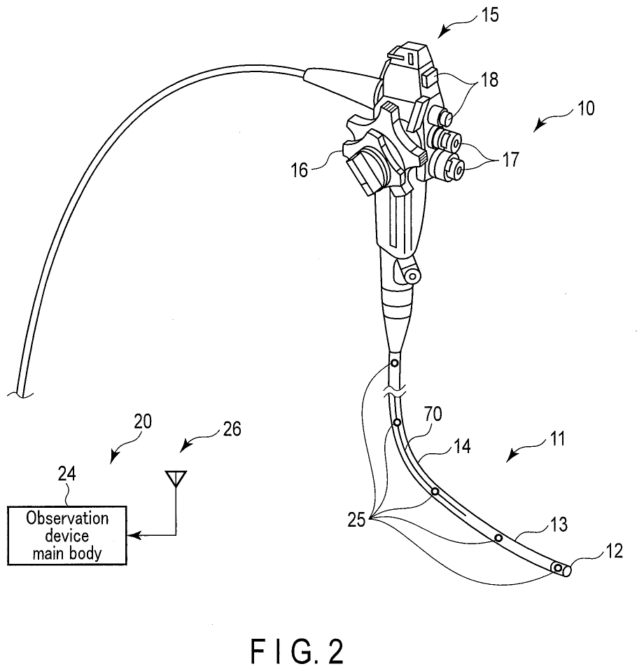

FIG. 2 is a diagram showing an example of the endoscope 10 and the insertion shape observation device 20. The endoscope 10 includes a tubular insertion section 11 to be inserted into an insertion target (here, the large intestine), and an operation portion 15 provided on the proximal end side of the insertion section 11. The insertion section 11 includes a distal end hard portion 12, a bendable section 13 connected to the proximal end side of the distal end hard portion 12, and a flexible tube section 14 connected to the proximal end side of the bendable section 13. The distal end hard portion 12 includes an illumination lens and an objective lens (not shown), and an imaging element 19 shown in FIG. 1. The bendable section 13 is a portion that is bent by the operation of the operation portion 15. The bending shape thereof can be actively changed. The flexible tube section 14 is an elongated tubular portion having flexibility, and bends passively. The operation portion 15 is provided with an angle knob 16 used for bending operation of the bendable section 13, and one or more buttons 17 used for various operations including air supply/water supply/suction operation. When an operator operates the angle knob 16, the bendable section 13 is bent in an arbitrary direction. In addition, the operation portion 15 is provided with one or more switches 18 to which functions such as stopping/recording and focus switching of endoscopic images are assigned according to the setting of the endoscope control device 40.

The endoscope 10 is connected to the light source apparatus 30. The light source apparatus 30 includes a light source such as a laser light source, an LED light source, a xenon lamp, and a halogen lamp. The light source apparatus 30 has a function of supplying illumination light to the illumination lens of the endoscope 10. Furthermore, the light source apparatus 30 may have a function of supplying air/water to a body cavity through the endoscope 10. The light source apparatus 30 includes, for example, an air supply pump for supplying air to the endoscope 10. The operator operates the button 17 of the operation portion 15 of the endoscope 10 to supply air from a nozzle (not shown) at the distal end of the endoscope or pressurize a water supply tank connected to the endoscope 10 to supply water from the nozzle at the distal end of the endoscope.

The endoscope apparatus 1 includes the insertion shape observation device 20. FIG. 1 and FIG. 2 show a magnetic sensor type insertion shape observation device 20 as an example. The insertion shape observation device 20 includes, for example, a controller 21, a receiver 22, and a transmitter 23 in an observation device main body 24. In addition, a plurality of source coils 25 provided in the insertion section 11 of the endoscope 10 and an antenna 26 separate from the observation device main body 24 also configure the insertion shape observation device 20.

As shown in FIG. 2, the insertion section 11 is provided with a plurality of source coils 25. The source coils 25 are magnetic field generating elements that generate a magnetic field. Each source coil 25 is disposed in the bendable section 13 and the flexible tube section 14 with an interval in the longitudinal direction in order to detect a bending shape in the longitudinal direction (axial direction) of the insertion section 11. FIG. 2 shows a form in which the source coil 25 is pre-installed in the insertion section 11; however, a probe with a built-in source coil may also be inserted into a channel extending in the longitudinal direction in the insertion section 11. Furthermore, the source coil 25 may be disposed on the entire flexible tube section 14 or only on a part thereof.

The antenna 26 is configured to detect a magnetic field generated by the source coil 25. The antenna 26 is separate from the observation device main body 24 and the endoscope 10, and is arranged around an insertion target into which the insertion section 11 of the endoscope 10 is to be inserted by a stand or the like (not shown).

The controller 21 of the insertion shape observation device 20 is a control circuit configured to execute various calculations in the insertion shape observation device 20 and to control display of the display device 60. The receiver 22 is a receiving circuit configured to receive a magnetic field detection signal from the antenna 26. The transmitter 23 is a transmitting circuit that sends out a drive signal to the source coil 25. The controller 21, in particular, performs calculation processing using a position estimation algorithm based on the magnetic field detection signal from the antenna 26.

The insertion shape observation device 20 is not limited thereto. The insertion shape observation device 20 may be any device configured to detect the bending state of the insertion section 11. For example, it may be configured by one of sensing using a change in the quantity of light (light intensity) propagating through a light guide member such as an optical fiber or a change in optical characteristics (fiber sensor), sensing using an electromagnetic wave (electromagnetic sensor), sensing using ultrasound (ultrasonic sensor), sensing using strain (strain sensor), and sensing using an X-ray absorbing material, or a combination thereof.

The flexible tube section 14 of the endoscope 10 is provided with a flexible tube characteristic changing unit 70 configured to partially change the bending stiffness of the flexible tube section 14 at a portion where it is provided. The flexible tube characteristic changing unit 70 may be, for example, a variable stiffness actuator whose hardness changes according to the voltage applied thereto from the endoscope control device 40. In FIG. 2, one flexible tube characteristic changing unit 70 is shown; however, the number of flexible tube characteristic changing units 70 is not limited thereto. That is, a plurality of flexible tube characteristic changing units 70 arranged along the longitudinal direction of the flexible tube section 14 may be provided.

The endoscope control device 40 includes a controller 41, a driver 42, and an image processor 43. Furthermore, the insertion support control device 100 includes an insertion state input section 111, an image input section 112, a situation determination section 113, and an information output section 114. The endoscope control device 40 and the insertion support control device 100 may be configured by a processor such as a CPU. That is, in the endoscope control device 40 and the insertion support control device 100, each of the above-described sections may be configured by a processor such as a CPU. In this case, for example, various programs for causing the processor to function as each section are prepared in an internal memory or an external memory (not shown). The processor executes the programs to perform the functions serving as each section of the endoscope control device 40 and the insertion support control device 100. Alternatively, each section of the endoscope control device 40 and the insertion support control device 100 may be configured by a hardware circuit, including an Application Specific Integrated Circuit (ASIC), Field-Programmable Gate Array (FPGA), and the like.

In the present embodiment, the above-described sections of the insertion support control device 100, that is, the insertion state input section 111, the image input section 112, the situation determination section 113, and the information output section 114, are included in the insertion support control device 100 that is separate from the endoscope control device 40 that is an endoscope video image processor. However, it is not limited thereto. That is, the processor or hardware circuit that functions as each of the above-described sections of the insertion support control device 100 may be included in one casing or may be included in a plurality of casings as long as it is able to perform the function of each section. This may also be included in the endoscope control device 40.

In the endoscope control device 40, the controller 41 is a control circuit that controls various operations of the endoscope 10. The driver 42 is a drive circuit configured to send out a signal for driving the imaging element 19 of the endoscope 10. The image processor 43 is an image signal processing circuit configured to convert an electrical signal converted from an optical signal by the imaging element 19 into a video signal. The controller 41 also performs light dimming control of the light source apparatus 30.

In the insertion support control device 100, bending shape information (bending angle, bending quantity, curvature, or radius of curvature, etc.) is input to the insertion state input section 111 from the insertion shape observation device 20 as the insertion state of the insertion section 11. Endoscopic image information is input from the endoscope control device 40 to the image input section 112. The situation determination section 113 determines the insertion situation of the insertion section 11 based on information from the insertion state input section 111 and the image input section 112. The information output section 114 generates insertion auxiliary information, that is, information corresponding to the operation, based on the determination by the situation determination section 113, and causes the display device 60 to display the generated information.

The input device 50 is a general input device such as a keyboard. Various commands for operating the endoscope apparatus 1 are input to the input device 50. The input device 50 may be an operation panel provided in the endoscope control device 40 or a touch panel displayed on a display screen. Although not shown, the observation device main body 24 and the insertion support control device 100 of the insertion shape observation device 20 are also provided with input sections such as an operation panel and a touch panel.

The display device 60 is a general monitor such as a liquid crystal display. The display device 60 displays the endoscopic image that has been subjected to image processing by the image processor 43 by the display control conducted by the controller 41 of the endoscope control device 40. The display device 60 may also display a three-dimensional image or character information related to the bending shape of the insertion section 11 based on the bending shape information from the insertion shape observation device 20 by the display control conducted by the controller 21 of the insertion shape observation device 20. The display device 60 also displays the insertion auxiliary information generated by the information output section 114 of the insertion support control device 100 by the display control conducted by the insertion support control device 100. The display device on which the endoscopic image is displayed, the display device on which the bending shape, etc. is displayed, and the display device on which the insertion auxiliary information is displayed may be the same or different. That is, the insertion auxiliary information may be displayed on a display device different from the display device 60.

Now, a general colonoscope insertion operation of the endoscope apparatus 1 will be explained.

In the endoscope apparatus 1, the insertion section 11 of the endoscope 10 is inserted from the anus into the rectum and colon by the operator. The endoscope 10 converts light from a subject in the intestinal tract into an electrical signal by the imaging element 19 of the distal end hard portion 12. The electric signal is then transmitted to the endoscope control device 40. The image processor 43 of the endoscope control device 40 acquires the electrical signal and converts the acquired electrical signal into a video signal. The controller 41 then causes the display device 60 to display an endoscopic image based on the video signal.

During insertion, the controller 21 of the insertion shape observation device 20 supplies drive signals having different frequencies from the transmitter 23 to the source coil 25. Thus, each source coil 25 generates a weak alternating magnetic field around it. That is, each source coil 25 outputs information related to the position thereof. The antenna 26 detects the magnetic field generated by the source coil 25 and sends out the detection signal to the receiver 22. The controller 21 calculates the position coordinates of each source coil 25 based on the magnetic field detection signal received by the receiver 22, generates, for example, a three-dimensional image of the insertion section 11 corresponding to the calculated position coordinates, and causes the display device 60 to display the generated image.

The colonoscope insertion procedure is difficult and takes time to acquire. For example, it is said that an operator needs experience of 1000 cases until the success rate of reaching the cecum with the distal end of the insertion section exceeds 90%. Furthermore, in the teaching of procedures, generally, it is necessary for a skilled physician (or a supervising physician) to attend the examination of an unskilled physician and give individual guidance. Unskilled physicians require time for conducting endoscopic examinations, which, sometimes, causes pain to the patient. In addition, there are time constraints on the skilled physician, too. Furthermore, there are many hospitals that do not necessarily have skilled physicians who can provide guidance.

In addition, in colonoscopy insertion, various insertion difficulties occur. For example, if an operator loses sight of the lumen direction to be inserted, the operator would need to find the lumen direction again, which would not be easy for an unskilled physician. Furthermore, the insertion section 11 inserted into the body cavity may be bent or may have various loops formed thereon, which may disturb the distal end of the insertion section from advancing even if the operator pushes the insertion section 11 from the hand side, or may cause the patient to feel pain by extending the intestinal wall by the force from the insertion section 11 applied thereto. For example, since the sigmoid colon is a movable intestinal tract that is not fixed in the abdomen, in particular, when a force that is applied from the insertion section 11 to the apex of the sigmoid colon (so-called S-top), which is the bent portion, causes hyperextension, the patient may be in pain.

In such a situation, it is effective to eliminate the bending of the insertion section 11 or resolve the loop shape (for example, so-called “straightening the scope”). However, it is difficult for an operator who is an unskilled physician to accurately determine the situation and perform an appropriate operation at an appropriate timing.

For example, in a colonoscopy insertion procedure, a method for inserting the insertion section 11 while folding the intestinal tract to be concentrically shortened and straightened is known. This method is called right turn shortening. Note that the shaft retention shortening method and hooking the fold are similar methods. In this method, the insertion section 11 is linearly advanced from the rectum to the S-top and becomes a cane shape when folded over the S-top and back. When the insertion section 11 is in a shape of a cane, the linear part thereof is called a rotation shaft. In the right turn shortening, the S-top is shortened by combining the operation of pulling out the insertion section 11 toward the hand side and the operation of twisting the rotation shaft clockwise, thereby straightening the sigmoid colon while being folded.

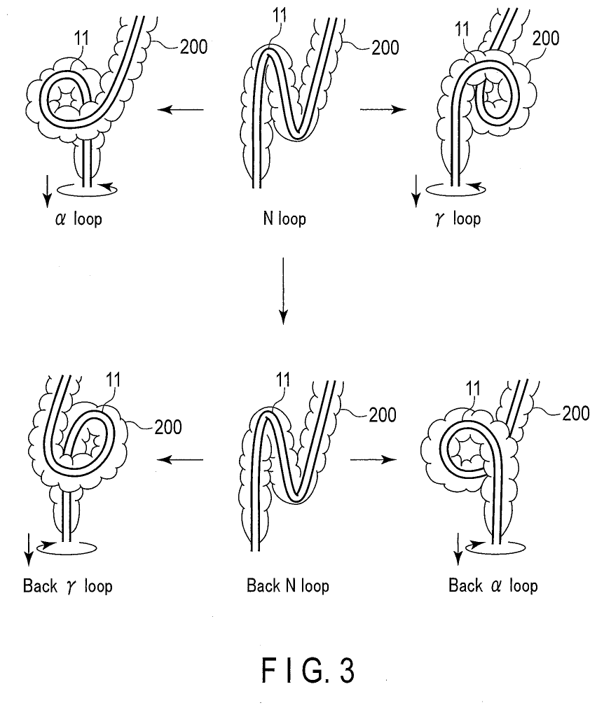

As shown in FIG. 3, in the intestinal tract 200 at the time of insertion, for example, the loop formation pattern in the sigmoid colon includes an α loop, an N loop, a γloop, and the like. The N loop of the right rotation shaft becomes the basis of performing insertion to the sigmoid colon by the above-mentioned right turn shortening. From this state, when the distal end of the insertion section flows to the left rotation shaft, an α loop is formed, and when it flows to the right rotation shaft, a γloop (reverse a loop) is formed. Even if the α loop and the γloop are formed, it is possible to proceed with the insertion while folding the sigmoid colon by resolving the loop formation by right turn shortening. Furthermore, a back N loop in which the N loop is inverted may be formed. From this state, a back γloop is formed when the distal end of the insertion section flows to the left rotation shaft, and a back α loop is formed when it flows to the right rotation shaft. In the case of the back loop, the loop formation is resolved by combining the operation of pulling out the insertion section 11 toward the hand side and the operation of twisting the rotation shaft counterclockwise. Therefore, in the case of the back loop, left turn & right turn shortening is performed, that is, after the loop formation is released by counterclockwise rotation, insertion proceeds while folding the sigmoid colon by clockwise rotation.

In the above manner, the colonoscopy insertion procedure varies depending on the loop formation pattern. Furthermore, α loop may occur not only on the sigmoid colon, but also on different sites such as on the transverse colon. Therefore, colonoscopy insertion procedures are diverse. With such a background, it is desired to shorten the time required for an unskilled physician to learn the colonoscopy insertion procedure, pursue efficiency thereof, and realize a safer endoscopic examination. In this regard, in the present embodiment, insertion support is provided by the insertion support control device 100.

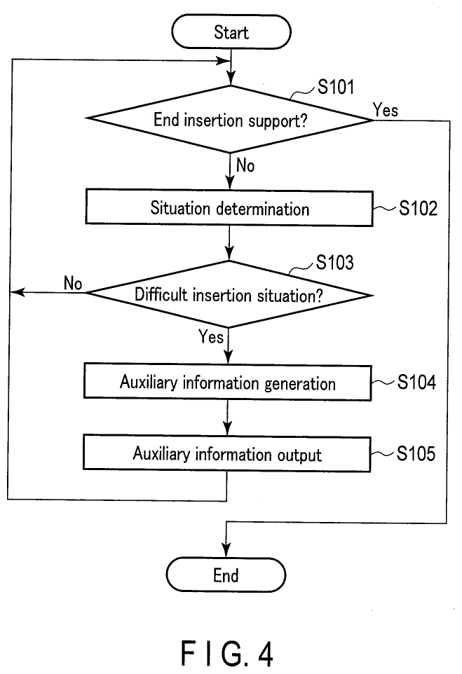

Hereinafter, an example of endoscope insertion support according to the present embodiment will be explained. FIG. 4 is a diagram showing an example of an insertion support flow according to the present embodiment.

In step S101, the insertion support control device 100 determines whether or not to end the insertion support, that is, whether or not an insertion support end instruction has been input. The end of the insertion support is determined based on an end instruction (input from the outside) based on, for example, the distal end of the insertion section reaching the cecum. In the case where the operator wishes to end the insertion support based on the endoscopic image acquired by the endoscope 10, the bent shape of the insertion section 11 acquired by the insertion shape observation device 20, or what is sensed by the operator's hand, etc., the operator may input an end instruction from an input section (not shown), etc. of the insertion support control device 100. In the case of determining that the insertion support be ended (Yes), the processing is ended. In the case of determining that the insertion support not be ended (No), the processing proceeds to step S102.

In step S102, situation determination is performed regarding the insertion situation of the insertion section 11. A plurality of endoscopic images generated in time series are input from the endoscope control device 40 to the image input section 112 of the insertion support control device 100. For example, the endoscope 10 captures an endoscopic image at 30 fps, and inputs the endoscopic image to the image input section 112 from the endoscope control device 40. The input endoscopic image is not limited to this, and may be captured at less than 30 frames per second by sampling. A predetermined number of input images, for example, 300 frames or more, is stored in an internal memory (or external memory) (not shown) provided in the image input section 112 or the situation determination section 113, or in the insertion support control device 100. The situation determination section 113 executes the situation determination of the endoscope insertion based on the endoscopic image input and stored in the image input section 112. For example, the situation determination section 113 performs the situation determination using an image of the past ten seconds, that is, 300 frames.

Furthermore, information related to the insertion state of the endoscope 10 may be input from the insertion shape observation device 20 to the insertion state input section 111 of the insertion support control device 100. The situation determination section 113 may execute the situation determination of the endoscope insertion based on the information related to the insertion state input to the insertion state input section 111 in addition to the endoscopic image input and stored in the image input section 112.

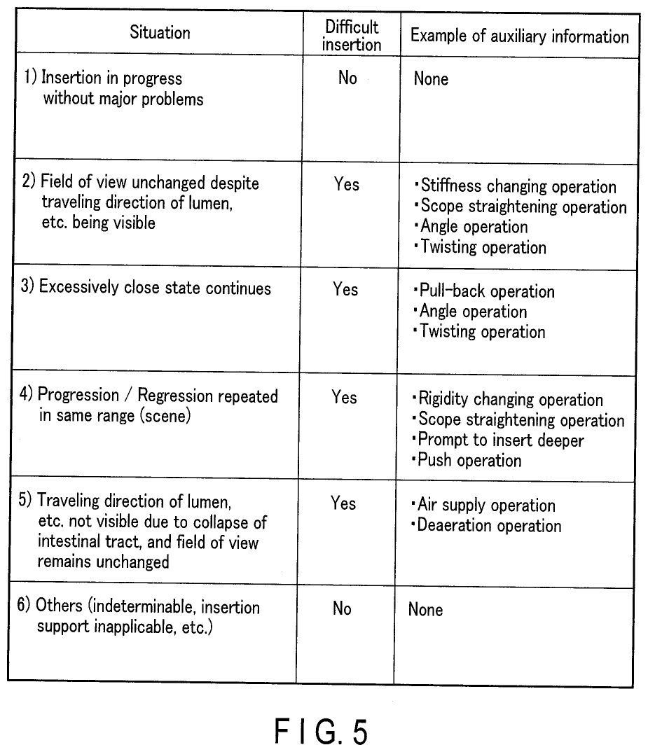

For example, in the insertion procedure of colonoscopy (excluding other actions such as observation, treatment, and therapy), situations shown in the left column of FIG. 5 may occur. The situation determination section 113 determines which situation has occurred based on the endoscopic image or the information related to the endoscopic image and the insertion state. For example, the situation determination section 113 executes the situation determination of the endoscope insertion based on the above-described 300-frame endoscopic image.

1) Insertion in Progress without Major Problems

When the situation determination section 113 detects that a plurality of endoscopic images (imaging scenes) input to the image input section 112 are continuously changing over a predetermined time, the insertion section 11 is determined as being inserted without any major problem. (Determination result 1). This is a situation where the insertion section 11 is proceeding smoothly through the intestinal tract. Alternatively, in addition to this, it may also be determined as Determination result 1 when the situation determination section 113 detects from the information related to the insertion state of the endoscope 10 that the insertion shape and insertion length of the insertion section 11 are continuously changing over a predetermined time.

2) Field of View Unchanged Despite Traveling Direction of Lumen, Etc. Being Visible

When the situation determination section 113 detects that a change in the image is minute (to an extent that the direction of the distal end of the insertion section is changed slightly) although a traveling direction of a lumen, etc. is visible in a plurality of endoscopic images input to the image input section 112, or detects that the image has hardly changed, it determines that the traveling direction of the lumen, etc. is visible but the field of view has not changed (Determination result 2). Alternatively, it may be determined as Determination result 2 when the situation determination section 113 detects that the change (movement quantity) on the distal end side of the insertion section 11 is small with respect to the operation (pushing operation or withdrawing operation) on the hand side of the insertion section 11 from the information related to the insertion state of the endoscope 10. In the case where the change on the distal end side of the insertion section 11 is small with respect to the pushing operation or the withdrawing operation on the hand side, it may be considered that the insertion section 11 is bent or a loop is formed. In addition, it may also be determined as Determination result 2 when the situation determination section 113 detects the bent portion itself or the loop itself from the information related to the insertion state of the endoscope 10.

3) Excessively Close State Continues

When the situation determination section 113 detects that a predetermined time (for example, 10 seconds) has passed in a state where a plurality of endoscopic images input to the image input section 112 remain excessively close to the mucous or in a reddish state, that is, for example, when the above 300-frame image is in a reddish state, the situation determination section 113 determines that an excessively close state is continuing (Determination result 3). The reddish state is a state in which the distal end of the insertion section is in contact with the mucous membrane and the entire image becomes a reddish color due to blood flow in the submucosal layer.

4) Progression/Regression Repeated in Same Range (Scene)

When the situation determination section 113 detects that an image substantially the same as that captured at time t is captured at time t+α in a plurality of time-series endoscopic images input to the image input section 112, the situation determination section 113 determines that progression/regression is being repeated in the same range (scene) (Determination result 4). This is a state in which the insertion section 11 has returned to the position and situation of the time t due to withdrawal, etc. For example, in a case where a certain scene substantially matches with an image captured within the past N frames, the situation determination section 113 determines such case as being Determination result 4.

5) Traveling Direction of Lumen, Etc. Not Visible Due to Collapse of Intestinal Tract, and Field of View Remains Unchanged

When the situation determination section 113 recognizes the collapse of the intestinal tract in a plurality of endoscopic images input to the image input section 112, and detects the change in the image as being minute (to an extent in which the direction of the distal end of the insertion section is slightly changed), or the image as being hardly changed, the situation determination section 113 determines that the traveling direction of the lumen, etc. is not visible due to the collapse of the intestinal tract, and that the field of view has not changed (Determination result 5).

6) Others (Indeterminable, Insertion Support Inapplicable, Etc.)

In the case where none of the above Determination results 1 to 5 applies, the situation determination section 113 determines such case as Determination result 6. This applies when the determination is impossible or the insertion support is inapplicable, etc.

As described above, among the Determination results 1 to 6, the Determination results 1 and 2 may be determination results based on an endoscopic image input to the image input section 112, determination results based on information related to an insertion state input to the insertion state input section 111, or determination results obtained by a combination thereof, and the Determination results 3 to 6 are determination results based on the endoscopic image input to the image input section 112. By using information related to the insertion state for the situation determination, it is possible to enhance recognition accuracy.

Regarding the above-described situation determination based on the endoscopic image, for example, detection of the quantity of change based on the correlation between the endoscopic images, scene recognition of each image, or a combination thereof can be used for the situation determination performed by the situation determination section 113. The situation determination section 113 performs situation determination by recognizing the same scene continuously or repeatedly, and the presence or absence of an important structure such as a lumen or folds, etc.

Furthermore, the situation determination section 113 may utilize a learning result using a deep neural network technology such as Recurrent Neural Network or 3D-CNN. As a result, by learning, the contents of such as the detection of the quantity of change based on the correlation between the endoscopic images and the scene recognition of each image can be recognized. The situation determination section 113 uses situation recognition (application of motion recognition) for a plurality of frame images using, for example, Recurrent Neural Network. The situation determination section 113 may perform determination by using an image sequence of a plurality of frames (for example, 30 frames×5 seconds=150 frames, or may be sampled at intervals of several frames) and recognition by learning based on categorized situations (categorizing Determination results 1 to 5). This may be a technique disclosed in Noriki Nishida, Hideki Nakayama, “Multimodal gesture recognition using multi-stream recurrent neural network”, Pacific-Rim Symposium on Image and Video Technology (PSIVT), 2015, or Self-Motion Identification by Convolutional-Recurrent Neural Network, Image Sensing Symposium, 2016 by Ryuji Kamiya et al.

In this manner, in step S102, the situation determination section 113 determines which one of the Determination results 1 to 6 mentioned above corresponds to the situation of the endoscope insertion based on a plurality of endoscopic images generated in time series input to the image input section 112, or based on this and information on the insertion state input to the insertion state input section 111.

In the subsequent step S103, the situation determination section 113 determines, based on the situation determination result (Determination results 1 to 6) in step S102, whether or not the operator encounters a difficult insertion situation, in other words, whether or not auxiliary information, that is, information corresponding to the operation, should be output to the operator. The middle column of FIG. 5 shows the determination result (Yes/No) of the insertion difficulty in step S103. In the case of Determination result 1, since insertion is performed without any major problem, it is not in a difficult insertion situation. Similarly, in the case of Determination result 6, it is also assumed as not being in a difficult insertion situation. Therefore, in the case of Determination results 1) and 6), the situation determination section 113 determines that the insertion situation is not difficult (No) in step S103, and returns to step S101. In the case of one of Determination results 2 to 5, the situation determination section 113 determines that the insertion situation is difficult (Yes), in which the insertion section 11 of the endoscope 10 cannot be successfully inserted even if insertion is continued, and proceeds to step S104.

In step S104, the information output section 114 generates auxiliary information corresponding to the situation determination result in step S102. For example, the information output section 114 includes a database in which information (for example, character information, image information, or a combination thereof) to be output is associated with respect to the determination result of the situation determination section 113, and selects the output information based on the determination result. That is, the information output section 114 outputs at least one auxiliary information from among a plurality of auxiliary information prepared in association with the endoscope insertion procedure based on the determination result by the situation determination section 113. In step S105, the information output section 114 causes the display device 60 to display the generated auxiliary information.

In the present embodiment, Determination results 2 to 5 present a situation where some kind of problem has occurred in the colonoscopy operation, and it is considered difficult for the operator to continue with the insertion without any support. In the following, typical problems and auxiliary information that may be effective in solving such problems, that is, support information, will be explained for each of the Determination results 2 to 5. This support information corresponds to, for example, an operation performed on the basis of experience when an operator who is experienced in colonoscopy encounters a difficult insertion situation. In the right column of FIG. 5, an example of auxiliary information in the Determination results 2 to 5 is shown.

Auxiliary Information in Determination Result 2

In the case where the traveling direction of the lumen, etc. is visible but the field of view does not change, a situation may be considered in which the pushing force is not transmitted to the distal end of the insertion section despite the operator pushing the insertion section 11 from the hand side. Therefore, for example, the information output section 114 generates auxiliary information that prompts the stiffness changing operation so that the flexible tube characteristic changing unit 70 increases the bending stiffness of the flexible tube section 14. When the bending stiffness of the flexible tube section 14 is increased, the flexible tube section 14 becomes difficult to bend, and thus the force with which the operator pushes the insertion section 11 from the hand side becomes easily transmitted to the distal end of the insertion section. Accordingly, the distal end of the insertion section can be easily advanced.

Alternatively, the information output section 114 generates auxiliary information that prompts the scope to be straightened. The auxiliary information that prompts the scope to be straightened may be a PULL operation in which the operator pulls back the insertion section 11 from the hand side, or α loop release operation as explained with reference to FIG. 3. With such auxiliary information, the operator performing a PUSH operation to push the insertion section 11 from the hand side switches the PUSH operation to the PULL operation or to the loop release operation. Thereby, the insertion situation can be improved.

Furthermore, even if the traveling direction of the lumen, etc. can be seen, a situation in which the distal end of the insertion section is not properly oriented in the lumen direction can be considered. Therefore, the information output section 114 derives a direction in which the bendable section 13 is to be angled, or a direction in which the insertion section 11 is to be twisted, and uses the derived direction as the auxiliary information. With such auxiliary information, the operator angles the bendable section 13 in the derived direction, or twists the insertion section 11 from the hand side. Thereby, the insertion situation can be improved.

Auxiliary Information in Determination Result 3

In the case where the distal end of the insertion section is excessively close to the mucosal surface, based on experience, a skilled physician would create a distance between the distal end of the insertion section and the mucosal surface by performing a PULL operation that once pulls back the insertion section 11 from the hand side, and would search for the lumen direction by operating the angle knob 16 of the operation portion 15 to operate the angle of the bendable section 13, or by twisting the insertion section 11 clockwise from the hand side. On the other hand, an unskilled physician may fear that the inserted insertion section 11 will come out, and may not be able to perform an appropriate PULL operation. In fact, even if the insertion section 11 comes out to some extent, re-insertion is often easy because the shape of the large intestine is relatively well suited to the insertion section 11 up to the site where it has been once inserted. Therefore, in the case of Determination result 3, it is useful to clearly prompt a PULL operation for pulling back the insertion section 11 as the support information. The information output section 114 generates auxiliary information that prompts the PULL operation. The operator performs the PULL operation based on such auxiliary information, thereby improving the insertion situation.

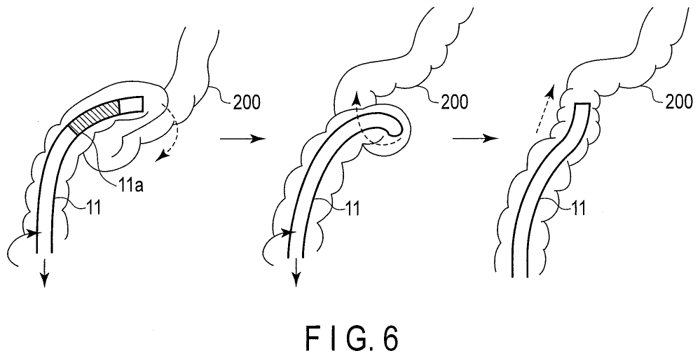

As shown in FIG. 6, for example, a non-loop method, in which the insertion section 11 advances without forming a loop, and without bending the intestinal tract 200, is known as one of the colonoscopy insertion methods. In this method, by twisting the insertion section 11 clockwise from the hand side, the bent portion 11a of the insertion section 11 transmits torque to the intestinal tract. Then, while pulling out the insertion section 11 slightly toward the hand side to change the way in which the insertion section 11 travels along the intestinal tract that is bent in a crank shape, the angle of the bendable section 13 is released without colliding with the mucous membrane. When the intestinal tract starts to rotate, and the angle is released in a manner that the insertion section 11 is not pulled out, the intestinal tract can be straightened. In the case of Determination result 3, for example, the information output section 114 may output auxiliary information so that such insertion by the non-loop method smoothly proceeds.

Auxiliary Information in Determination Result 4

In the case of repeating progression/regression in the same range (scene), in the same manner as Determination result 2, the force by which the insertion section 11 is pushed from the hand side may not be sufficiently transmitted to the distal end of the insertion section. Therefore, it is useful to prompt changing the bending stiffness or straightening the scope. For example, the information output section 114 generates auxiliary information that prompts the stiffness changing operation so that the bending stiffness of the flexible tube section 14 is increased by the flexible tube characteristic changing unit 70. As a result, the flexible tube section 14 becomes difficult to bend, and the force with which the operator pushes the insertion section 11 from the hand side is transmitted to the distal end of the insertion section, so that the distal end of the insertion section can easily advance. Alternatively, the information output section 114 generates auxiliary information that prompts the scope to be straightened (a pull back operation or α loop release operation, etc.). The operator performs the operation based on such auxiliary information, thereby improving the insertion situation.

For example, when performing the insertion by the above-mentioned right turn shortening, in some cases, the operation for shortening/straightening the intestinal tract may not be performed successfully, thereby, repeating the similar progression/regression in the same range (scene). This also corresponds to Determination result 4. In this case, for example, the information output section 114 generates auxiliary information that prompts the operator to try an operation for shortening and straightening the intestinal tract after inserting the insertion section 11 deeper, or generates auxiliary information that prompts the operator to give up the shortening/straightening operation and switch to the insertion by the PUSH operation. The operator performs the operation based on such auxiliary information, thereby improving the insertion condition.

Auxiliary Information in Determination Result 5

In a situation where the lumen is collapsed, an operation for appropriately expanding the lumen by air supply (or water supply) to the endoscope 10 may be required. On the other hand, it is known that if the intestinal tract is expanded by excessive air supply, the subsequent insertion becomes more difficult. Therefore, while prompting the expansion of the lumen by air supply, it is desirable to stop the air supply when an appropriate quantity of air supply has made the lumen recognizable.

Therefore, in the case of Determination result 5, for example, the information output section 114 generates auxiliary information that prompts the lumen to be expanded by air supply and, subsequently, stops the air supply when an appropriate air supply quantity is reached and the lumen is made recognizable. The operator performs the operation based on such auxiliary information, thereby improving the situation where the lumen is collapsed. It is also useful to promote the deaeration of excess air after the lumen has expanded and the insertion section 11 has passed. The information output section 114 may generate auxiliary information that prompts deaeration after the insertion section 11 has passed. As a result, it is possible to make the insertion easier to proceed after the collapsed state of the lumen is improved.

As noted in Determination result 5, the auxiliary information generated or selected by the information output section 114 in each of the Determination results 2 to 5 is not limited to one. The information output section 114 may generate or select a plurality of auxiliary information. The plurality of auxiliary information may be simultaneously output or sequentially output to the display device 60.

After step S105, the process returns to step S101, and the insertion support control device 100 determines whether or not the insertion support is to be ended. In the case of determining that the insertion support is to be ended (Yes), the processing is ended. In the case of determining that the insertion support is not to be ended (No), the processing repeats step S102 and the subsequent steps.

As explained above, in the present embodiment, the information output section 114 outputs at least one auxiliary information from among a plurality of auxiliary information prepared in association with the colonoscopy insertion procedure based on the determination result by the situation determination section 113. The colonoscopy insertion procedure includes a method of advancing the distal end of the insertion section mainly by a PUSH operation along the bent shape of the intestinal tract (so-called loop insertion method), or a method of shortening the intestinal tract while retaining the longitudinal axis of the insertion section and inserting it (so-called shaft retention shortening method; the above-mentioned right turn shortening is categorized in this method), etc. In the present embodiment, the information output section 114 can select and output appropriate auxiliary information from among a plurality of auxiliary information prepared in association with these procedures according to the determination result of the insertion situation.

According to the present embodiment, when an operator encounters a difficult situation in inserting an endoscope, it is possible to output support information according to the content of the difficulty in a timely manner. Therefore, even in the absence of a supervising physician, by providing appropriate support information to the operator, a smoother endoscopic examination can be performed, and the skill required for endoscope insertion can be assisted.

In particular, upon colonoscopy insertion, since a movable intestinal tract such as the sigmoid colon easily moves, and, further, has a bent portion such as an S-top, an operator with little experience may find insertion to be difficult. In contrast, in the present embodiment, various difficult events assumed in colonoscopy insertion and methods to deal with them are associated with each other and prepared in advance in the database of the information output section 114 as support information. Therefore, timely assistance and support can be provided when the assumed difficult event occurs. By the operator performing colonoscopy with appropriate assistance and support, the patient's pain caused by excessive force applied to the intestinal tract from the insertion section 11 may be reduced, and the examination time may be shortened.

In addition to the determination based on a plurality of time-series endoscopic images input to the image input section 112 and the determination based on this and information on the insertion state input to the insertion state input section 111, the situation determination section 113 may perform situation determination by utilizing detection information detected by various sensors such as a sensor configured to detect a movement quantity by the PUSH operation or the PULL operation of the insertion section 11, the sensor configured to detect the clockwise or counterclockwise rotation quantity of the insertion section 11, the sensor configured to detect the bending quantity of the bendable section 13 by an angle operation of the angle knob 16, or the sensor configured to detect the force applied to the insertion section 11. This allows situation determination to be performed with higher accuracy, and allows accurate support information to be provided.

For example, in a case where a plurality of sensors configured to detect the quantity of force received by the insertion section 11 from the outside (for example, the intestinal tract) are arranged along the longitudinal direction of the insertion section 11, the situation determination section 113 can use the quantity of force detected by each sensor in addition to the endoscopic image information for determination. By the determination in such manner, in the case of Determination result 2 (the traveling direction of the lumen, etc. is visible but the field of view has not changed), it can be more accurately determined whether or not this is caused by loop formation.

In addition, the auxiliary information output by the information output section 114 is not limited to visual information, and may be auditory auxiliary information such as sound emitted from a speaker (not shown), etc., or a combination of visual auxiliary information and auditory auxiliary information.

Moreover, the situation determined by the situation determination section 113 is not limited to a state in which insertion is difficult. For example, the situation determination section 113 may detect and determine a push operation or an angle operation that is too fast, etc. using the above-described sensor, etc. In this case, the auxiliary information output by the information output section 114 is information for suppressing such operation.

In the above, an endoscope apparatus including a colonoscope has been explained; however, the present invention is not limited thereto. The concept of the present invention can also be applied to a lower digestive tract endoscope or an upper digestive tract endoscope other than the colonoscope. The concept of the present invention is also applicable to an endoscope apparatus other than an endoscope apparatus provided with a medical endoscope, or a flexible tube insertion device.

The present invention is not limited to the above embodiments, and can be modified in various manners in practice when implementing the invention without departing from the gist of the invention. Moreover, each of the embodiments may be implemented by being suitably combined to the extent possible, in which case a combined effect will be obtained. Furthermore, the above embodiments include inventions at various stages, and various inventions can be extracted by appropriately combining a plurality of disclosed constituent elements.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An insertion support device comprising:
an image input section to which a plurality of endoscopic images generated in time series are input;
a situation determination section configured to determine a situation of endoscope insertion based on the plurality of inputted endoscopic images; and
an information output section configured to output information corresponding to at least one operation from information corresponding to a plurality of operations prepared in association with an endoscope insertion procedure, based on a determination result by the situation determination section.

2. The insertion support device according to claim 1, wherein
the situation determination section is configured to determine a situation of the endoscope insertion based on image change information in the plurality of endoscopic images.

3. The insertion support device according to claim 1, wherein
the plurality of endoscopic images are endoscopic images generated by colonoscopy, and
the situation determination section is configured to determine an insertion situation of the colonoscopy.

4. The insertion support device according to claim 1, wherein
the determination result by the situation determination section includes a determination result as to whether or not an insertion difficulty event has occurred in the endoscope insertion, and
the information output section is configured to output information corresponding to at least one operation corresponding to an insertion difficulty event, when the situation determination section determines that the insertion difficulty event has occurred.

5. The insertion support device according to claim 1, wherein
the information output section is configured to output at least one of information related to an operation of an endoscope and information related to a direction in which the endoscope is to be inserted as information corresponding to the operation.

6. The insertion support device according to claim 1, further comprising
an insertion state input section to which information related to an insertion state of an endoscope is input, wherein
the information output section is configured to determine a situation of the endoscope insertion based on information related to the insertion state in addition to the plurality of endoscopic images.

7. The insertion support device according to claim 6, wherein
the information related to the insertion state includes at least one of information related to an insertion shape of an insertion section of the endoscope and information related to an insertion length of the insertion section of the endoscope.

8. The insertion support device according to claim 1, wherein,
the information output section is configured to output information prompting a stiffness changing operation, a straightening operation, an angle operation, or a twisting operation of an insertion section of an endoscope as information corresponding to the operation, when the situation determination section determines that a direction of travel is visible but a field of view does not change.

9. The insertion support device according to claim 1, wherein,
the information output section is configured to output information prompting a pull-back operation, an angle operation, or a twist operation of an insertion section of an endoscope as information corresponding to the operation, when the situation determination section determines that an excessively close state continues.

10. The insertion support device according to claim 1, wherein,
the information output section is configured to output information prompting a stiffness changing operation, a straightening operation, an angle operation, or a twisting operation of an insertion section of an endoscope as information corresponding to the operation, when the situation determination section determines that progression and regression are repeated in a same range.

11. The insertion support device according to claim 1, wherein,
the information output section is configured to output information prompting an air supply operation or a deaeration operation of an endoscope as information corresponding to the operation, when the situation determination section determines that a direction of travel of a lumen is not visible and the field of view does not change.

12. An insertion support method comprising:
inputting a plurality of time-sequentially generated endoscopic images;
determining a situation of endoscope insertion based on the plurality of inputted endoscopic images; and
outputting information corresponding to at least one operation from information corresponding to a plurality of operations prepared in association with an endoscope insertion procedure, based on the determining the situation.

13. The insertion support method according to claim 12, wherein
the determining the situation includes determining a situation of an endoscope insertion based on image change information in the plurality of endoscopic images.

14. The insertion support method according to claim 12, wherein
the plurality of endoscopic images are endoscopic images generated by colonoscopy, and
the determining the situation includes determining an insertion situation of the colonoscopy.

15. The insertion support method according to claim 12, wherein
a determination result obtained by the determining the situation includes a determination result as to whether or not an insertion difficulty event has occurred in the endoscope insertion, and
the outputting information corresponding to the operation includes outputting information corresponding to at least one operation corresponding to an insertion difficulty event when the determining the situation determines that the insertion difficulty event has occurred.

16. The insertion support method according to claim 12, wherein
the outputting information corresponding to the operation includes outputting at least one of information related to an operation of an endoscope and information related to a direction in which the endoscope is to be inserted as information corresponding to the operation.

17. The insertion support method according to claim 12, further comprising
inputting information related to an insertion state of an endoscope, wherein
the outputting information corresponding to the operation includes determining a situation of an endoscope insertion based on information related to the insertion state in addition to the plurality of endoscopic images.

18. The insertion support method according to claim 17, wherein
the information related to the insertion state includes at least one of information related to an insertion shape of an insertion section of the endoscope and information related to an insertion length of the insertion section of the endoscope.

19. An endoscope apparatus comprising:
the insertion support device according to claim 1; and
an endoscope having a bendable section configured to be actively bent, and an insertion section that is connected to a proximal end side of the bendable section and includes a flexible tube section configured to be passively bent.
